(19) United States
(12) Patent Application Publication
(10) Pub. No.: US 2026/0128172 A1
(43) Pub. Date: May 7, 2026

(54) INFORMATION PROCESSING METHOD, INFORMATION PROCESSING APPARATUS, INFORMATION PROCESSING RECORDING MEDIUM, METHOD FOR GENERATING TRAINED MODEL, TRAINED MODEL, AND MEASUREMENT DEVICE

(71) Applicant: OMRON HEALTHCARE Co., Ltd., Kyoto (JP)

(72) Inventors: Hideyuki KOBAYASHI, Kyoto (JP); Yoshihiro SUESE, Kyoto (JP)

(21) Appl. No.: 19/427,428

(22) Filed: Dec. 19, 2025

Related U.S. Application Data
(63) Continuation of application No. PCT/JP2024/036153, filed on Oct. 9, 2024.

(30) Foreign Application Priority Data
Dec. 25, 2023 (JP) 2023-218152

(51) Int. Cl. G16H 50/20; G16H 10/60

(52) U.S. Cl. CPC G16H 50/20; G16H 10/60

(57) ABSTRACT

An information processing method, an information processing apparatus, and an information processing program that are capable of determining the presence or absence of a symptom of a respiratory disease with high accuracy, a trained model used for these information processing method, apparatus, and program, a method for generating the trained model, and a measurement device. A processor performs processing for acquiring data based on measurement data from a subject and indicating a respiratory state of the subject, acquiring medication history information related to the subject and exercise history information related to the subject in a period prior to a measurement timing of the measurement data, inputting the data, the medication history information, and the exercise history information to a trained model, and obtaining a determination result on the presence or absence of a symptom of a respiratory disease in the subject from the trained model.

### CROSS-REFERENCE TO RELATED APPLICATIONS

This application is the U.S. national stage application filed pursuant to 35 U.S.C. 365(c) and 120 as a continuation of International Patent Application No. PCT/JP2024/036153, filed October 9, 2024, which application claims priority to Japanese Patent Application No. 2023-218152, filed December 25, 2023, which applications are incorporated herein by reference in their entireties.

### FIELD

The technology of the present disclosure relates to an information processing method, an information processing apparatus, an information processing program, a method for generating a trained model, a trained model, and a measurement device.

### BACKGROUND

Patent Document 1 describes a treatment support device including a processor that acquires measurement sound data of a lung sound measurement device that is attached to the body of a subject and measures lung sound, acquires action data of the subject in a measurement period of the measurement sound data, and displays an image based on the measurement sound data and the action data on a display device of an external device.

Patent Document 2 discloses a treatment support device including a processor which acquires determination result data of a device which determines an airway state of a subject during a measurement period of a lung sound based on the lung sound measured from the subject, and state information indicating a state of the subject in a period different from the measurement period in a day including the measurement period, and records the determination result data and the state information in association with each other.

### CITATION LIST

### Patent Literature

Patent Document 1: JP 2022-016972 A

Patent Document 2: JP 2021-194272 A

### SUMMARY

### Technical Problem

The presence or absence of symptoms of a respiratory disease such as asthma, pulmonary fibrosis, upper airway obstruction, or chronic obstructive pulmonary disease is determined with high accuracy by a doctor directly examining a patient using a stethoscope or the like. On the other hand, devices such as a peak flow meter and a sensor that can detect wheezing are also known, and if such a device is used, it is possible to determine the presence or absence of symptoms without having to see a doctor. The determination of the presence or absence of a symptom by the device is performed using breath sound, vital capacity, or the like measured from the patient. However, since the state of the body of a patient varies, it is desirable to determine the presence or absence of a symptom in consideration of the state.

An object of the technology of the present disclosure is to provide an information processing method, an information processing apparatus, and an information processing program that are capable of determining the presence or absence of a symptom of a respiratory disease with high accuracy, and to provide a trained model used for these information processing method, apparatus, and program, a method for generating the trained model, and a measurement device.

### Solution to Problem

The technique of the present disclosure is as follows. Note that components and the like corresponding to those in the following embodiments are indicated in parentheses, but the components are not limited thereto.

(1) An information processing method including: by a processor (processor 11),acquiring data (processed measurement data) based on measurement data measured from a subject (user) and indicating a respiratory state of the subject; acquiring action history information related to an action history of the subject in a period prior to a measurement timing of the measurement data; and inputting the data and the action history information to a trained model (trained model 13) and obtaining a determination result on the presence or absence of a symptom of a respiratory disease in the subject from the trained model.

(2) The information processing method according to (1), in which the action history information includes at least one of medication history information related to a medication history or exercise history information regarding an exercise history.

(3) The information processing method according to (2), in which the medication history information includes the number of times of medication in the period.

(4) The information processing method according to (2) or (3), in which the exercise history information includes information on an amount of exercise.

(5) The information processing method according to claim (2) or (3), in which

the exercise history information includes information on a frequency at which an exercise has been performed under a specific condition.

(6) An information processing apparatus (information processing server 10) including: a processor (processor 11) configured to: acquire data (processed measurement data) based on measurement data measured from a subject (user) and indicating a respiratory state of the subject; acquire action history information related to an action history of the subject in a period prior to a measurement timing of the measurement data; and input the data and the action history information to a trained model (trained model 13) and obtain a determination result on the presence or absence of a symptom of a respiratory disease in the subject from the trained model.

(7) An information processing recording medium causing a processor (processor 11) to execute: acquiring data (processed measurement data) based on measurement data measured from a subject (user) and indicating a respiratory state of the subject; acquiring action history information related to an action history of the subject in a period prior to a measurement timing of the measurement data; and inputting the data and the action history information to a trained model (trained model 13) and obtaining a determination result on whether or not a symptom of a respiratory disease has appeared in the subject from the trained model.

(8) A method for generating a trained model, the method including:

by a processor (processor 11), acquiring, as training data, a plurality of pieces of data based on measurement data measured from a first subject (monitor user) and indicating a respiratory state of the first subject, a plurality of pieces of action history information related to an action history of the first subject in a period prior to a measurement timing of the measurement data, and a plurality of determination results by a doctor on whether or not a symptom of a respiratory disease has appeared in the first subject on a day including the measurement timing; and causing a recording medium (or program) to execute machine learning based on the plurality of pieces of training data to obtain a trained model (trained model 13) that outputs a determination result on whether or not a symptom of a respiratory disease has appeared in a second subject (user) upon an input of data (processed measurement data) based on measurement data measured from the second subject and indicating a respiratory state of the second subject, and action history information related to an action history of the second subject in a period prior to a measurement timing of the measurement data.

(9) A trained model (trained model 13) having undergone machine learning using, as training data, data (processed measurement data) based on measurement data measured from a first subject (monitor user) and indicating a respiratory state of the first subject, action history information related to an action history of the first subject in a period prior to a measurement timing of the measurement data, and a determination result by a doctor on whether or not a symptom of a respiratory disease has appeared in the first subject on a day including the measurement timing, the trained model (trained model 13) causing a processor (processor 11) to: output a determination result on whether or not a symptom of a respiratory disease has appeared in a second subject (user) upon an input of data (processed measurement data) based on measurement data measured from the second subject and indicating a respiratory state of the second subject, and action history information related to an action history of the second subject in a period prior to a measurement timing of the measurement data.

(10) A measurement device (measurement device 40) configured to measure measurement data indicating a respiratory state of a subject from the subject, the measurement device including: a communicator configured to communicate with a terminal (user terminal 30) capable of acquiring action history information related to an action history of the subject in a period prior to a measurement timing of the measurement data; an output device; and a processor, wherein the processor is configured to: transmit the measurement data to the terminal; input data based on the measurement data and the action history information to a trained model, and receive, from the terminal, a determination result on the presence or absence of a symptom of a respiratory disease in the subject obtained from the trained model; and cause the output device to output the received determination result.

### Advantageous Effects of Invention

According to the technology of the present disclosure, it is possible to provide an information processing method, an information processing apparatus, and an information processing program capable of determining the presence or absence of a symptom of a respiratory disease with high accuracy, a trained model used therefor, a method for generating the trained model, and a measurement device.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic diagram illustrating a schematic configuration of a symptom determination system 100.

### DESCRIPTION OF EMBODIMENTS

### Outline of Information Processing Method According to Technology of Present Disclosure

In the information processing method, a processor performs a process of acquiring data based on measurement data indicating a respiratory state of a subject measured from the subject, acquiring action history information (for example, at least one of medication history information related to a medication history or exercise history information regarding an exercise history) related to an action history of the subject in a period prior to a measurement timing of the measurement data, inputting the data and the action history information to a trained model, and obtaining a determination result on the presence or absence of a symptom of a respiratory disease in the subject from the trained model.

In this manner, by inputting not only the data based on the measurement data measured from the subject, but also the action history information based on the past actions of the subject to the trained model, it is possible to obtain the determination result on the presence or absence of symptoms of respiratory disease in the subject from the output of the trained model. As a result, compared to a configuration in which the presence or absence of a symptom is determined by analyzing measurement data measured from the subject using a specific algorithm, as with a wheezing sensor or the like, it is possible to determine the presence or absence of a symptom that takes into consideration the past actions of the subject, and it is possible to increase the determination accuracy.

For example, it is assumed that the subject takes a therapeutic drug prescribed by a doctor. When the subject determines the presence or absence of a symptom using the device, it is assumed that a result indicating that there is no symptom is output from the device in a case where the airway state has been appropriately improved by medication. On the other hand, when the subject is examined by a doctor in the same state, the doctor observes the breath sound or the like while inquiring about the medication status or the like in order to ascertain the effect of the medication, and thus it is assumed that the doctor makes a stricter determination than the device so as not to miss a slight sign of the symptom. In this manner, there may be a difference in criteria between the determination by the device and the determination by the doctor depending on the medication history of the subject. Further, the airway state of the subject can be changed not only by the medication history but also by the action history. For example, since stress tends to be reduced in a person who frequently performs exercise, it is assumed that the airway state is improved. However, in this case as well, depending on the contents of the action history of the subject, the determination result of the doctor may differ from the determination result of the device.

In the technique of the present disclosure, the presence or absence of a symptom is determined by a trained model that has performed machine learning using, as training data, action history information acquired from each of a large number of persons (monitor users to be described later), data based on measurement data indicating a respiratory state, and a determination result on the presence or absence of a symptom by a doctor's examination. Therefore, it is possible to determine the presence or absence of a symptom in consideration of not only the data based on the measurement data conventionally used by the device but also the behavior history (medication history, action history, and the like). As a result, a result close to the determination by the doctor can be obtained from the trained model, and it is possible to determine the presence or absence of the symptom of the respiratory disease with high accuracy.

Hereinafter, a configuration example of a respiratory disease symptom determination system including a device that executes an information processing method according to the technology of the present disclosure will be described.

### System Configuration

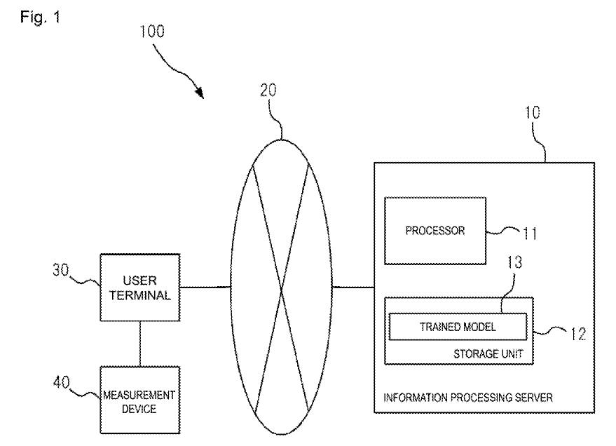

FIG. 1 is a schematic diagram illustrating a schematic configuration of a symptom determination system 100. The user who uses the symptom determination system 100 is a user who has been diagnosed with a respiratory disease by a doctor and has received a prescription of a therapeutic drug, but even a user who has not received a prescription of a therapeutic drug can use the symptom determination system 100. The symptom determination system 100 includes an information processing server 10, a user terminal 30 such as a smartphone possessed by a user (subject), and a measurement device 40 possessed by the user. The user terminal 30 and the information processing server 10 are connected to a network 20 such as the Internet and are configured to be able to communicate with each other.

The measurement device 40 is a device for measuring measurement data indicating the respiratory state of the user from the user. The measurement data indicating the user's respiratory state is, for example, a breath sound (e.g., data of time vs. amplitude value) or a vital capacity data (e.g., data of expiratory volume vs. flow volume (flow-volume curve)).

The measurement device 40 can be, for example, a device that includes a sensor including a microphone and measures and records breath sound data of the user by the microphone in a state in which the sensor is applied to the chest of the user. The measurement device 40 can also be a peak flow meter that measures and records the user's flow-volume curve. The measurement device 40 and the user terminal 30 can be connected by wired communication or wireless communication, and measurement data measured by the measurement device 40 is transmitted to the user terminal 30. Specifically, the measurement device 40 includes a sensor (a flow amount detector in the case of a peak flow meter), a communicator capable of communicating with the user terminal 30 in a wireless or wired manner, an output device such as a display or a speaker, and a device processor that integrally controls these components.

A management application capable of managing the action history of the user is installed in the user terminal 30. The management application records medication history data indicating a medication history which is one of the exercise histories of the user and action history data indicating an action history which is one of the exercise histories in the memory. The management application records medication history data and action history data of the user using a position detection function, an activity amount detection function, an input function of various kinds of information, an acquisition function of various kinds of information from an external server via the network 20, and the like mounted on the user terminal 30.

When the user takes the therapeutic drug prescribed by the doctor, the user operates the management application to input the date and time when the drug was taken. Accordingly, the management application records the input information in the memory as medication history data.

When the user exercises while carrying the user terminal 30 or wearing a smartwatch or the like that can be connected to the user terminal 30, the action history data of the user is acquired and recorded in the memory by the management application. The action history data of the user includes, for example, the intensity of the exercise (unit: METs) when the exercise has been performed, the period during which the exercise has been performed, the place where the exercise has been performed (either indoors or outdoors), and the environment of the place where the exercise has been performed (temperature, allergen amount, or the like). The location and the environment indicate a situation in which exercise is performed. The action history data of the user may include an activity amount such as the number of steps or calorie consumption for each unit period (for example, one day). The amount of allergen is defined by, for example, the amount of scattered pollen or PM 2.5.

The intensity of exercise, the number of steps, calorie consumption, and the like can be acquired by an activity amount detection function of the user terminal 30 or a smart watch. The place where the exercise has been performed can be acquired by a position detection function of the user terminal 30 or a smartwatch. The environment of the place where the exercise has been performed can be acquired by an acquisition function of various kinds of information of the user terminal 30.

The information processing server 10 includes a processor 11 and a storage unit 12. The storage unit 12 is configured to include, for example, a non-transitory storage medium such as a hard disk or flash memory in addition to a working memory such as a random access memory (RAM). The storage unit 12 stores an information processing program for the processor 11 of the information processing server 10 to execute an information processing method.

The processor 11 is, for example, a central processing unit (CPU) that is a general-purpose processor configured to execute software (program) and perform various functions, a programmable logic device (PLD) that is a processor whose circuit configuration can be changed after manufacturing, such as a field programmable gate array (FPGA), or a dedicated electric circuit that is a processor including a circuit configuration specifically designed to execute specific processing, such as an application specific integrated circuit (ASIC). The processor 11 may include one processor, or may include a combination of the same type or different types of two or more processors (for example, a plurality of FPGAs or a combination of a CPU and an FPGA). More specifically, the hardware structure of the processor 11 is an electric circuit (circuitry) in which circuit elements such as semiconductor elements are combined. In a case where the processor 11 is configured with a plurality of processors, the plurality of processors do not need to be installed in the same device, and may be provided in a plurality of devices distributed via the network 20.

The storage unit 12 stores a trained model 13. The trained model 13 is generated by causing a training model configured by a program to execute machine learning using teaching data (training data). The trained model 13 may be generated by the processor 11 of the information processing server 10 or may be generated by a processor of a computer different from the information processing server 10. Hereinafter, a method of generating the trained model 13 will be described assuming that the processor 11 generates the trained model 13.

### Method for Generating Trained Model

The processor 11 acquires the sample data group from the storage unit 12. Each sample data included in the sample data group includes medication history data and action history data (hereinafter, referred to as first data) recorded over a predetermined period (for example, one week, two weeks, one month, or the like) by a monitor user different from the user who uses the symptom determination system 100 using the same terminal as the user terminal 30. In addition, each sample data includes measurement data (hereinafter, referred to as second data) obtained by the monitor user measuring the measurement data indicating the respiratory state using the same device as the measurement device 40 after the end of the predetermined period (for example, the next day of the day when the predetermined period ends). In addition, each sample data includes a determination result (hereinafter, third data) of the presence or absence of a symptom of a respiratory disease by a doctor when the monitor user receives a medical examination by the doctor on a day including the timing at which the measurement data is measured. In this manner, the sample data including the first data, the second data, and the third data is generated for each of a large number of monitor users, and is recorded in the storage unit 12 as a sample data group.

The processor 11 generates medication history information related to a medication history of the monitor user in a predetermined period based on the first data in each sample data of the sample data group acquired from the storage unit 12. The medication history information is information obtained by processing the medication history data, and is, for example, the number of times of medication (that is, medication frequency) during a predetermined period, the number of times of medication for each divided period when the predetermined period is divided into a plurality of periods (information indicating a distribution of timings at which medication has been performed in the predetermined period), or the like.

Further, the processor 11 generates exercise history information related to the exercise history of the monitor user in a predetermined period based on the first data in each sample data of the sample data group acquired from the storage unit 12. The exercise history information is information obtained by processing the exercise history data and includes, for example, first information concerning an amount of exercise during a predetermined period or second information concerning the frequency of exercise performed under a specific condition during the predetermined period. The exercise history information may be configured by a combination of the first information and the second information.

As the first information, for example, an amount (METs ・ hour) of physical activity of a predetermined intensity (for example, 3 METs) or more is used. For example, in a case where an exercise with an intensity of 3 METs has been performed for one hour and an exercise with an intensity of 5 METs has been performed for one hour during the predetermined period, (3 METs × 1 hour) + (5 METs × 1 hour) = 8 [METs × hour] is generated as the first information on the amount of exercise. As the first information on the amount of exercise, the number of times or a time during which a physical activity with a predetermined intensity or more has been performed during a predetermined period may be generated. As the first information on the amount of exercise, a cumulative number of steps, a cumulative calorie consumption, or the like during a predetermined period may be generated.

As the second information, for example, indoor exercise information indicating the number of times or a time in which exercise with predetermined intensity or more has been performed indoors during a predetermined period and outdoor exercise information indicating the number of times or a time in which exercise with predetermined intensity or more has been performed outdoors during a predetermined period are generated.

The outdoor exercise information may be further subdivided. For example, the outdoor exercise information may be generated by being divided into first outdoor exercise information indicating the number of times or a time in which exercise of a predetermined intensity or more has been performed in a state in which the amount of allergen is a predetermined level or more and second outdoor exercise information indicating the number of times or a time in which exercise of a predetermined intensity or more has been performed in a state in which the amount of allergen is less than the predetermined level.

In addition to or instead of the first outdoor exercise information and the second outdoor exercise information, third outdoor exercise information indicating the number of times or a time during which exercise of a predetermined intensity or more has been performed in a state in which the temperature is a predetermined level or more, and fourth outdoor exercise information indicating the number of times or a time during which exercise of a predetermined intensity or more has been performed in a state in which the temperature is less than the predetermined level may be generated. Indoor, outdoor, outdoor with an allergen amount equal to or higher than a predetermined level, outdoor with an allergen amount lower than a predetermined level, outdoor with a temperature equal to or higher than a predetermined level, and outdoor with a temperature lower than a predetermined level each constitute a specific condition.

Further, based on the second data (measurement data) in each sample data of the sample data group acquired from the storage unit 12, the processor 11 generates processed measurement data obtained by processing the second data into a format suitable for learning.

As the processed measurement data, a feature value extracted from the measurement data (breath sound data or vital capacity data) is used. As the feature value, a value that can cause a significant difference between a person who does not have a respiratory disease and a person who has a respiratory disease can be appropriately adopted.

In a case where the measurement data is breath sound data, for example, the processor 11 generates data of frequency vs. amplitude value from the breath sound data (data of time vs. amplitude value), selects a plurality of predetermined frequencies (frequencies at which a difference in amplitude between a person with a respiratory disease and a person without a respiratory disease is particularly large) in this data, and obtains an amplitude value corresponding to each of the frequencies as processed measurement data.

Alternatively, the processor 11 may process the breath sound data to generate data of frequency vs. sound pressure, and acquire, as the processed measurement data, a ratio between a peak value of the sound pressure in the data and a width between frequencies corresponding to local minimum values of the sound pressure on both sides of the peak value.

Alternatively, the processor 11 may process the breath sound data to generate three dimensional data of a frequency, a sound pressure, and a time, and select a frequency in a period in which the sound pressure has a maximum value in the three dimensional data as the processed measurement data. In addition, the processor 11 may obtain a feature value (for example, the number of times and the time when the amplitude becomes equal to or greater than a certain value) that can be directly extracted from the breath sound data as the processed measurement data without performing the frequency analysis on the breath sound data.

It is known that there is a difference in the shape of the vital capacity (flow-volume curve) measured by a peak flow meter between a person without a respiratory disease and a person with a respiratory disease. Therefore, when the measured data is the vital capacity data, the processor 11 extracts, from the flow-volume curve as the vital capacity data, a flow amount (for example, a flow amount corresponding to 50% or 25% of the maximum value of the expiratory volume) that can specify the difference in the shape, thereby obtaining the flow amount as the processed measurement data.

Alternatively, the processor 11 may extract, from the flow-volume curve, flow amounts corresponding to a plurality of ratios (for example, 90%, 80%, and 70%) with respect to the maximum value of the expiratory volume, and use the flow amounts as the processed measurement data.

Alternatively, the processor 11 generates, from the vital capacity data, data in which the horizontal axis represents the absolute exhaust volume and the vertical axis represents the flow amount. In this data, it is known that there is a difference in the center of gravity position of the absolute exhaust volume between a person without a respiratory disease and a person with a respiratory disease. Therefore, the processor 11 may specify the center of gravity value of the absolute exhaust volume from the data of the absolute exhaust volume vs. the flow amount, and set the center of gravity value as the processed measurement data.

The processor 11 generates the trained model 13 by causing a training model to execute machine learning based on a plurality of data sets corresponding to a plurality of monitor users using a data set of the medication history information, the exercise history information, and the processed measurement data corresponding to the monitor user generated as described above and the third data (the determination result on the presence or absence of the symptom by the doctor) corresponding to the monitor user as teaching data. Each of the medication history information and the exercise history information constitutes action history information related to an action history.

The trained model 13 has learned various parameters so as to output a determination result on the presence or absence of a symptom of a respiratory disease in a user when medication history information and exercise history information in a predetermined period and processed measurement data generated based on measurement data measured from the user after the predetermined period are input. The machine learning method is not particularly limited. For example, any of methods such as logistic regression, a decision tree, random forests, a gradient boosting decision tree, and a neural network can be used.

### Use of Trained Model

First, the user measures the measurement data by the measurement device 40 in a state where the user terminal 30 and the measurement device 40 are connected. The measurement data measured by the measurement device 40 and the measurement date and time information thereof are transmitted from the measurement device 40 to the user terminal 30 and acquired by the management application of the user terminal 30. When the measurement data is acquired, the management application reads the medication history data and the action history data recorded in the past period from the measurement date and time (measurement timing) of the measurement data to the predetermined period before from the memory, and transmits the medication history data and the action history data to the information processing server 10 together with the measurement data.

When the processor 11 acquires the medication history data, the action history data, and the measurement data transmitted from the user terminal 30, the processor 11 generates medication history information on the basis of the medication history data, generates exercise history information on the basis of the exercise history data, and generates processed measurement data on the basis of the measurement data.

Next, the processor 11 inputs the generated medication history information, the exercise history information, and the processed measurement data to the trained model 13, and acquires the determination result on the presence or absence of the symptom of the respiratory disease as an output of the trained model 13. Upon acquiring the determination result, the processor 11 transmits the determination result to the management application of the user terminal 30. The management application performs a process of displaying the received determination result on the display device of the user terminal 30 or outputting the received determination result by voice from the speaker. This allows the user to know the presence or absence of a symptom. Alternatively, the management application transmits the received determination result to the measurement device 40. When the determination result is received, the device processor of the measurement device 40 outputs the determination result from the output device. Accordingly, the determination result may be output from the display or the speaker of the measurement device 40. By performing the measurement to the result output by the measurement device 40, the value of the measurement device 40 can be increased.

### Variation of Trained Model

The trained model 13 is obtained by performing machine learning using the data set of the medication history information, the exercise history information, the processed measurement data, and the third data (the determination result by the doctor) as teaching data, but is not limited thereto.

For example, the trained model 13 may be obtained by performing machine learning using a data set of any one of the medication history information or the exercise history information, the processed measurement data, and the third data (the determination result by the doctor) as teaching data. In this case, the processor 11 may input any one of the medication history information or the exercise history information of the user and the processed measurement data to the trained model 13 and obtain the determination result on the presence or absence of the symptom from the trained model 13.

The trained model 13 may be trained by machine learning so as to output a determination result on the presence or absence of a symptom of a respiratory disease using at least one of medication history information, exercise history information, information on a sleep history of the user (for example, a sleep time per day during a period or an average value of sleep scores during the period), information on a drinking history of the user (for example, a drinking amount per day during the period or the number of times of drinking during the period), information on a smoking history of the user (for example, the number of cigarettes smoked per day during the period or the number of cigarettes smoked during the period), or information on a bathing history of the user (for example, a bathing time per day during the period or the number of times of bathing during the period) and processed measurement data as inputs. Each of the sleep history, the drinking history, the smoking history, and the bathing history is one of the behavior histories of the user.

Machine learning may be executed by further including user information in the above-described data set.

The user information is information related to attributes (gender, age, physique (BMI), nationality, etc.) of the monitor user who is the measurement source of the measurement data included in the sample data. The processor 11 generates, for example, the medication history information, the exercise history information, and the processed measurement data based on each sample data of the sample data group, uses these pieces of information, the user information, and the third data as teaching data, and causes the learning program to execute machine learning based on the teaching data to generate the trained model 13. As a result, it is possible to generate the trained model 13 that outputs the determination result on the presence or absence of the symptom of the respiratory disease in the user when the medication history information, the exercise history information, the processed measurement data, and the user information for any user are input. In this manner, by further using the user information, it is possible to determine the presence or absence of the symptom of the user with higher accuracy.

In the above description, the processor 11 generates the medication history information, the exercise history information, and the processed measurement data, but there is no limitation thereto. For example, the terminal-side processor that executes the management application of the user terminal 30 may generate the medication history information, the exercise history information, and the processed measurement data, and transmit the generated medication history information, exercise history information, and processed measurement data to the information processing server 10, so that the processor 11 may acquire the medication history information, the exercise history information, and the processed measurement data. In a case where the terminal-side processor generates the medication history information, the exercise history information, and the processed measurement data, the trained model 13 may be incorporated into the management application. In this case, the terminal-side processor inputs the medication history information, the exercise history information, and the processed measurement data to the trained model 13 to obtain the determination result on the presence or absence of the symptom.

Although various embodiments are described above, it will be obvious that the present invention is not limited to such examples. It will be apparent to those skilled in the art that various changes and modifications can be made within the scope of the claims, and it is understood that these naturally belong to the technical scope of the present invention. In addition, components of the above-described embodiments may be combined as appropriate without departing from the spirit of the invention.

This application is based on Japanese Patent Application No. 2023-218152 filed on Dec. 25, 2023, the contents of which are incorporated herein by reference.

### REFERENCE SIGNS LIST

10 Information processing server

11 Processor

12 Storage unit

13 Model

20 Network

30 User terminal

40 Measurement device

100 Symptom determination system

What is claimed is:

1. An information processing method comprising:
by a processor,
acquiring data based on measurement data measured from a subject and indicating a respiratory state of the subject;
acquiring action history information related to an action history of the subject in a period prior to a measurement timing of the measurement data; and
inputting the data and the action history information to a trained model, and obtaining a determination result on presence or absence of a symptom of a respiratory disease in the subject from the trained model.

2. The information processing method according to claim 1, wherein the action history information includes at least one of medication history information related to a medication history or exercise history information related to an exercise history.

3. The information processing method according to claim 2, wherein the medication history information includes the number of times of medication in the period.

4. The information processing method according to claim 2, wherein the exercise history information includes information on an amount of exercise.

5. The information processing method according to claim 3, wherein the exercise history information includes information on an amount of exercise.

6. The information processing method according to claim 2, wherein the exercise history information includes information on a frequency at which an exercise has been performed under a specific condition.

7. The information processing method according to claim 3, wherein the exercise history information includes information on a frequency at which an exercise has been performed under a specific condition.

8. An information processing apparatus comprising
a processor configured to:
acquire data based on measurement data measured from a subject and indicating a respiratory state of the subject;
acquire action history information related to an action history of the subject in a period prior to a measurement timing of the measurement data; and
input the data and the action history information to a trained model, and obtain a determination result on presence or absence of a symptom of a respiratory disease in the subject from the trained model.

9. An information processing recording meaning causing a processor to execute:
acquiring data based on measurement data measured from a subject and indicating a respiratory state of the subject;
acquiring action history information related to an action history of the subject in a period prior to a measurement timing of the measurement data; and
inputting the data and the action history information to a trained model, and obtaining a determination result on whether or not a symptom of a respiratory disease has appeared in the subject from the trained model.

10. A method for generating a trained model, the method comprising:
by a processor,
acquiring, as training data, a plurality of pieces of data based on measurement data measured from a first subject and indicating a respiratory state of the first subject, a plurality of pieces of action history information related to an action history of the first subject in a period prior to a measurement timing of the measurement data, and a plurality of determination results by a doctor on whether or not a symptom of a respiratory disease has appeared in the first subject on a day including the measurement timing; and
causing a recording medium to execute machine learning based on the plurality of pieces of training data to obtain a trained model that outputs a determination result on whether or not a symptom of a respiratory disease has appeared in a second subject upon an input of data based on measurement data measured from the second subject and indicating a respiratory state of the second subject, and action history information related to an action history of the second subject in a period prior to a measurement timing of the measurement data.

11. A trained model having undergone machine learning using, as training data, data based on measurement data measured from a first subject and indicating a respiratory state of the first subject, action history information related to an action history of the first subject in a period prior to a measurement timing of the measurement data, and a determination result by a doctor on whether or not a symptom of a respiratory disease has appeared in the first subject on a day including the measurement timing, the trained model causing a processor to:
output a determination result on whether or not a symptom of a respiratory disease has appeared in a second subject upon an input of data based on measurement data measured from the second subject and indicating a respiratory state of the second subject, and action history information related to an action history of the second subject in a period prior to a measurement timing of the measurement data.
